(19) United States
(12) Patent Application Publication
(10) Pub. No.: US 2015/0173674 A1
(43) Pub. Date: Jun. 25, 2015

(54) DETECTING AND COMMUNICATING HEALTH CONDITIONS

(71) Applicant: Diabetes Sentry Products Inc., Orono, MN (US)

(72) Inventors: Timothy A. Hayes, Fort Worth, TX (US); Michael Russin, Orono, MN (US)

(73) Assignee: Diabetes Sentry Products Inc., Orono, MN (US)

(21) Appl. No.: 14/136,986

(22) Filed: Dec. 20, 2013

(51) Int. Cl. A61B 5/00; A61B 5/1455; A61B 5/11; A61B 5/01; A61B 5/145

(52) U.S. Cl. CPC A61B 5/681; A61B 5/4266; A61B 5/01; A61B 5/14532; A61B 5/7278; A61B 5/7282; A61B 5/7475; A61B 5/746; A61B 5/0008; A61B 5/1112; A61B 5/112; A61B 5/14551; A61B 5/6898; A61B 5/002

(57) ABSTRACT

This disclosure provides devices, systems, and methods for detecting physiological parameters, and for communicating information related to the detected physiological parameters to the user, the user's caregiver, and medical personnel. For example, some systems described herein can be configured to detect a user's hypoglycemic event, and to thereafter alert the user, caregivers, and medical personnel to the occurrence of the hypoglycemic event.

### TECHNICAL FIELD

This document relates to systems and methods for detecting physiological parameters using, for example, a wearable sensor device configured for communicating information related to the detected physiological parameters to the user, the user's caregiver, and medical personnel.

### BACKGROUND

Hypoglycemia is a condition characterized by abnormally low blood glucose levels. Humans need a certain amount of sugar (e.g., glucose) in order to function properly. If an individual's blood glucose level becomes too low, as occurs with hypoglycemia, it can cause confusion, abnormal behavior, double vision and blurred vision, seizures, loss of consciousness, brain damage, or even death. Physiological symptoms of hypoglycemia may include sweating, chills, heart palpitations, shakiness, anxiety, hunger, and tingling sensations around the mouth.

Early detection and treatment of hypoglycemic events is desirable. When detected early, the individual experiencing hypoglycemia can take relatively simple remedial actions such as consuming fast-acting carbohydrates, glucose tablets, or candy, and discontinuing insulin intake. As untreated hypoglycemia progresses, it becomes more difficult to correct. Corrective actions may then require the involvement of another individual such as a caregiver or medical personnel. Therefore, to manage hypoglycemia effectively, individuals experiencing hypoglycemia and, in some situations, caregivers, can benefit from being alerted to the early detection of symptoms of hypoglycemia.

### SUMMARY

This document describes devices, systems, and methods for detecting physiological parameters, and for communicating information related to the detected physiological parameters to the user, the user's caregiver, and medical personnel. For example, this document provides devices, systems, and methods for detecting a hypoglycemic condition occurring in a user of a wearable sensor device, and for alerting the user, a caregiver, medical personnel, or a combination thereof to the occurrence of such a hypoglycemic event.

In some embodiments, a wearable physiological sensing device may include a housing that is configured to be worn by a user such that a portion of the housing is in contact with a skin surface of the user. The sensing device may also include a perspiration sensor system disposed at least partially within the housing and configured to detect a level of perspiration on the skin surface of the user. The sensing device may optionally include a temperature sensor system disposed at least partially within the housing and configured to detect a temperature of the skin surface of the user. The sensing device may also include control circuitry disposed at least partially within the housing. The control circuitry may be configured to detect a hypoglycemic event of the user in response to receiving sensor information from the perspiration sensor system indicative of the level of perspiration on the skin surface of the user, from the temperature sensor system indicative of the temperature of the skin surface of the user, or both. The sensing device may further include a user interface attached to the housing and configured to an output alarm when the control circuitry detects the hypoglycemic event of the user. Additionally, the sensing device may include a wireless communication device disposed at least partially within the housing and configured to wirelessly communicate with one or more external devices.

Particular embodiments described herein may include a wearable physiological sensing and response system. The system may include a wearable physiological sensing device, which may include a housing, a physiological sensor, and a wireless communication device. The housing may be configured to be worn by a user such that a portion of the housing is in contact with a skin surface of the user. The physiological sensor may be positioned at least partially within the housing and configured to detect a physiological parameter of the user via contact with the skin surface of the user. The wireless communication device disposed at least partially within the housing and configured to wirelessly communicate with one or more external devices. The system may further include a computing device that is separate from the wearable physiological sensing device. The computing device may be configured to wirelessly communicate with the wireless communication device such that the computing device is configured to provide notifications of physiological alarm events in response to receiving an alarm event communication from the wireless communication device of the wearable physiological sensing device.

In further embodiments, a method for monitoring a user's physiological parameters includes arranging a wearable physiological sensing device in contact with a skin surface of the user. The wearable physiological sensing device of this method may include a housing that is configured to be worn by the user such that a portion of the housing is in contact with the skin surface of the user. The wearable physiological sensing device of this method may also include a perspiration sensor system disposed at least partially within the housing and configured to detect a level of perspiration on the skin surface of the user. The wearable physiological sensing device of this method may further include a temperature sensor system disposed at least partially within the housing and configured to detect a temperature of the skin surface of the user. The wearable physiological sensing device of this method may also include a wireless communication module disposed at least partially within the housing and configured to send and receive wireless communications to and from one or more other devices. The method may further include monitoring the level of perspiration on the skin surface of the user and the temperature of the skin surface of the user. The method may also include determining, based on the monitored level of perspiration on the skin surface of the user and the monitored temperature of the skin surface of the user, whether a physiological alarm condition exists. The method may also include initiating, based on a determination that a physiological alarm condition exists, a first alarm notification at the wearable physiological sensing device, and sending a wireless alarm event communication from the wireless communication module. The method may further include receiving, at a second device that is not connected to the wearable physiological sensing device, the alarm event communication sent from the wireless communication module. The method may also include providing, in response to receiving the alarm event communication sent from the wireless communication module, a second alarm notification at the second device.

In some embodiments described herein, a wearable physiological sensing device may include a wearable housing including an exterior surface configured to contact with a skin surface of the user. Also, the sensing device may include at least one physiological parameter sensor positioned at least partially within the housing and configured to detect physiological parameter via the skin surface of the user. The sensing device may further include control circuitry disposed at least partially within the housing and configured to detect a health alarm event of the user in response to receiving sensor information from the physiological parameter sensor. The sensing device may also include a user interface attached to the housing and configured to an output alarm when the control circuitry detects the health alarm event. The sensing device may further include a wireless communication device mounted at least partially within the housing and configured to wirelessly communicate with one or more external devices.

In particular embodiments, a system includes a wearable physiological sensing device and a computing device that is separate from the wearable physiological sensing device. The wearable physiological sensing device may include a wearable housing including an exterior surface configured to contact with a skin surface of the user. The wearable physiological sensing device may also include at least one physiological parameter sensor positioned at least partially within the housing and configured to detect physiological parameter via the skin surface of the user. The wearable physiological sensing device may further include a wireless communication device mounted at least partially within the housing and configured to wirelessly communicate with one or more external devices. The wearable physiological sensing device may be configured to detect a health alarm event of the user in response to receiving sensor information from the physiological parameter sensor. In this system, the computing device may be configured to wirelessly communicate with the wireless communication device. Optionally, the computing device may output notifications of physiological alarm events in response to receiving an alarm event communication from the wireless communication device of the wearable physiological sensing device.

Some or all of the embodiments described herein may provide one or more of the following advantages. First, in some embodiments, a user of a system for monitoring physiological parameters can be promptly alerted to the symptoms of a health event, such as a hypoglycemic event, so that the user may take timely remedial actions. The system may include a wearable physiological sensing device that is configured to provide such alerts, to wirelessly communicate with one or more external devices to provide such alerts, or a combination thereof.

Second, in some situations notifications of health events may also be communicated to a designated caregiver, a remote monitoring station (e.g., a health clinic for tracking user physiological parameters), or an emergency call center. In particular embodiments, the conditions for sending such notifications can be configured in accordance with user and/or caregiver preferences, and such communications can be sent in a progressive sequence.

Third, in some embodiments the user of a system for monitoring physiological parameters can provide supplemental information that is received and stored at a remote health monitoring station. Such information can append the recorded physiological data to enhance the later analysis of the recorded data.

Fourth, in some embodiments the wearable physiological sensing device can communicate and interact with other health monitoring systems, such as a continuous blood glucose sensor, a blood glucose meter (e.g., a test strip device), or a wearable insulin infusion pump system. As such, information from the wearable physiological sensing device can be output to the user via one or more of these health monitoring systems.

Fifth, in some embodiments, a caregiver or medical personnel can receive an indication from the wearable physiological sensor device of whether the wearable physiological sensing device is presently being worn by the user. In such cases, alert messages can be communicated to a caregiver if the device is not being worn, for example, by a user during the nighttime hours when some hypoglycemic events might otherwise go undetected and untreated.

Sixth, some embodiments of the wearable physiological sensing device can be equipped with a GPS transponder or other location-tracking device. As such, a caregiver can receive information from the wearable physiological sensing device indicative of the location of the user of the physiological sensing device. Such information may be useful, for example, when the caregiver is located remotely from the user of the physiological sensing device.

Seventh, some embodiments of the wearable physiological sensing device can be equipped with one or more complementary devices and systems such as accelerometers or other motion sensors, an oximetry system, graphene sensors, a pedometer, a smart watch device, and the like system. These complementary devices can be used by the wearable physiological sensing device to output additional contextual information to the user, a caregiver, medical personnel, or a combination thereof.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of a system including a system and other external devices, in accordance with some embodiments provided herein.

FIG. 2 is a perspective view of the wearable physiological sensing device of FIG. 1 and schematically depicts the elements that are included in the device, in accordance with some embodiments.

FIG. 3 is a front view of some computing devices that can be included in the system of FIG. 1 and schematically depicts elements that are included in the computing devices, in accordance with some embodiments.

FIG. 4 a perspective view of a docking station and wearable physiological sensing device of FIG. 1, in accordance with some embodiments.

FIG. 5 is a perspective view of the wearable physiological sensing device of FIG. 1 in communication with an external computing device, in accordance with some embodiments.

FIGS. 6A-6D illustrate example screen shots of the external computing device in communication with the wearable physiological sensing device of FIG. 5, in accordance with some embodiments.

FIG. 7 is a diagram that depicts an example process by which the system of FIG. 1 can operate in accordance with some embodiments.

FIG. 8 is a flowchart that depicts an example process implemented by a wearable physiological sensing device, in accordance with some embodiments.

Like reference numbers represent corresponding parts throughout.

### DETAILED DESCRIPTION OF ILLUSTRATIVE EMBODIMENTS

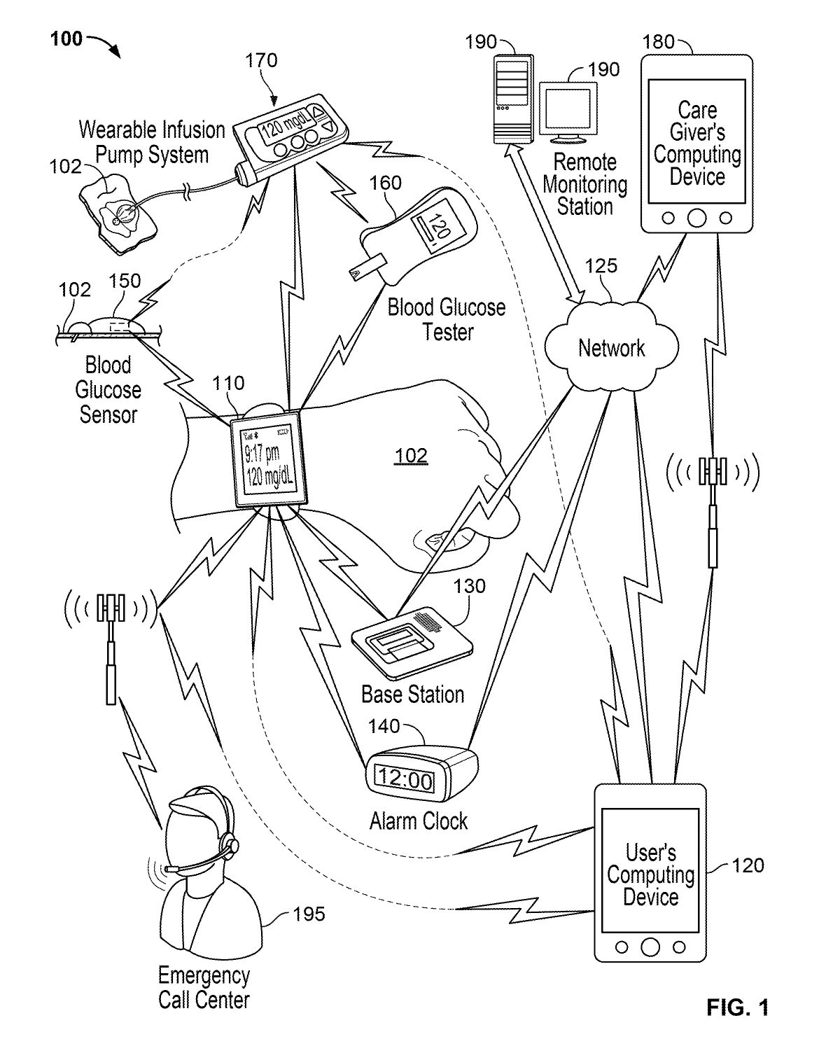

Referring to FIG. 1, some embodiments of a system 100 include a wearable physiological sensing device 110 that is in contact with a skin surface 102 of a user, and that can wirelessly communicate with other devices and systems. For example, in this particular embodiment, the wearable physiological sensing device 110 can be implemented in the form factor of a wristwatch such that a rear surface of the main body is maintained in contact with the skin surface 102 of the user. As will be described further below, the wearable physiological sensing device 110 can monitor one or more physiological parameters of the user, and can wirelessly communicate information relating to the monitored parameters to the other external devices and systems. The other types of devices and systems in communication with the wearable physiological sensing device 110 is dependent on the configuration of the system 100. For example, in some embodiments the system 100 can optionally include one or more of a user's computing device 120, a base station 130, an alarm clock 140, a continuous blood glucose sensor device 150, a blood glucose meter (BGM) device 160, a portable infusion pump system 170, a caregiver's computing device 180, a remote monitoring station 190, user interface devices within a vehicle, and other similar devices. However, such external devices are optional, and some embodiments of the system 100 may none of, or only a subset of, these aforementioned external devices and systems.

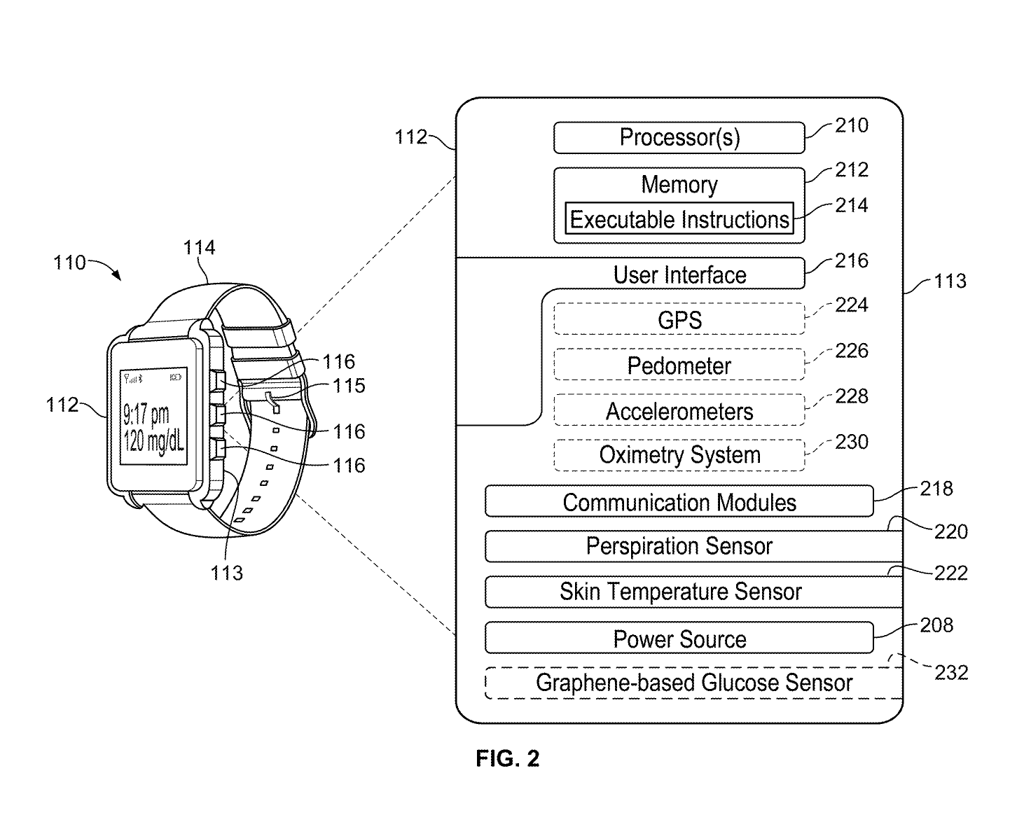

The wearable physiological sensing device 110 can monitor one or more physiological parameters of the user such as, but not limited to, skin temperature, perspiration, oxygen saturation levels in the blood, pulse rate, movement, and blood glucose levels. In particular embodiments, sensor elements for monitoring such parameters can be positioned at least partially in a housing 112 (FIG. 2) of the wearable physiological sensing device 110, and may be at least partially exposed along an exterior rear surface 113 (FIG. 2) of the housing 112 for positioning adjacent to the user's skin 102 (e.g., the user's wrist in this example embodiment). While in this embodiment the wearable physiological sensing device 110 is being worn on the user's wrist, wearing the sensing device 110 on the wrist location is not required in all implementations. For example, the wearable physiological sensing device 110 (or another sensing device embodiment with similar functionality but with a different form factor) can be located on other portions of the user's body including, but not limited to, the user's ankle, arm, leg, torso, forehead, and neck.

In particular embodiments, the sensing device 110 may receive communications from one or more ancillary devices that monitor the user's physiological parameters and/or other user attributes. For example, in this embodiment the sensing device 110 may receive communications that indicate a blood glucose level of the user from the continuous blood glucose sensor 150, the blood glucose meter device 160 (e.g., a test strip device), or the wearable insulin infusion pump system 170. In addition, in some embodiments the sensing device 110 may send communications to the ancillary devices to initiate certain tasks by the ancillary devices. For example, in the depicted embodiment the sensing device 110 may optionally send a communication to the wearable infusion pump system 170 to suspend insulin dispensations in response to a hypoglycemia event. The sensing device 110 may also send and receive communications from the user's computing device 120. For example, the communications sent from the user's computing device 120 to the sensing device 110 can include configuration of operational settings, alarm acknowledgements, data inquiries, event notifications, and the like.

In addition to monitoring the one or more physiological parameters, the wearable physiological sensing device 110 can include algorithms and memory-stored threshold values for determining the existence of an alarm condition in relation to the monitored physiological parameter(s). In one such example, a hypoglycemia alarm condition may be determined based on a drop in the monitored skin temperature over a predefined period of time, in comparison to a threshold value for a skin temperature rate-of-change. If the algorithm of the sensing device 110 finds that the monitored skin temperature has changed at a rate that exceeds the threshold value, the sensing device 110 thereby determines that an alarm condition exists. When the wearable physiological sensing device 110 has determined that an alarm condition exists, the wearable physiological sensing device 110 can initiate a local alarm via one or more user interface components (visual display alert, audible alert, vibratory alert, or a combination thereof) of the sensing device 110 and may optionally wirelessly communicate with one or more of the external devices 120, 130, 140, 150, 160, 170, 180 (via the network 125), and 190 (via the network 125) of the system 100 to prompt a remedial response to the detected alarm condition, and to record the occurrence of the alarm condition.

The wearable physiological sensing device 110 can communicate with some of the external devices 120, 130, 140, 150, 160, 170, 180 (via the network 125), and 190 (via the network 125) using various wireless communication modes. For example, in some embodiments the wearable physiological sensing device 110 can house one or more wireless communication devices configured to communicate using short-range wireless communication modes. Examples of such short-range communication modes implemented by the sensing device 110 can include, but are not limited to, infrared (IR), radio frequency (RF), Wi-Fi, Bluetooth, ANT+, radio-frequency identification (RFID), near-field communications (NFC), IEEE 802.15.4, and IEEE 802.22. In addition, in some embodiments the wearable physiological sensing device 110 can house one or more wireless communication devices configured to communicate with some of the external devices using various types of long-range wireless modes. Examples of such long-range communication modes implemented by the sensing device 110 can include, but are not limited to, cellular communications, network communications (e.g., internet, intranet, telephone networks, broadband phone service, broadband networks, wide area networks, and local area networks).

One example scenario which illustrates some operations of the physiological sensing and response system 100 will now be described. The wearable physiological sensing device 110 may periodically measure one or more physiological parameters. The measured parameter may be used as an input to a program being run by control circuitry (including one or more computer processors) housed within the main body 112 of the device 110. In some embodiments, the control circuitry receives the input (physiological parameter inputs) and, in response thereto, outputs a determination whether a health alarm condition exists, and such determination may be based on whether a predefined threshold limit value or range is exceeded (e.g., greater than an upper limit, less than a lower limit, or the like). If an alarm condition is determined to exist, in some embodiments a local alarm at the wearable physiological sensing device 110 is initiated. Such local alarms may include auditory alarms, visual alarms, tactile alarms, and combinations thereof. The alarm may optionally include an indication of the severity level of the alarm status (e.g., a high severity level alarm may be indicated visually using red color, or may be indicated audibly using a particular alarm tone or volume, and the like). In particular embodiments, the local alarm emitted from the sensing device 110 has a magnitude (visually, audibly, vibratory, or a combination thereof) that is sufficient to awake the user from a sleeping state. In some embodiments, the user of the wearable physiological sensing device 110 may be able to interact with the user interface 216 (e.g., using a touchscreen, one or more depressible buttons, a microphone, or a combination thereof) acknowledge and silence the local alarm via the user interface of the wearable physiological sensing device 110.

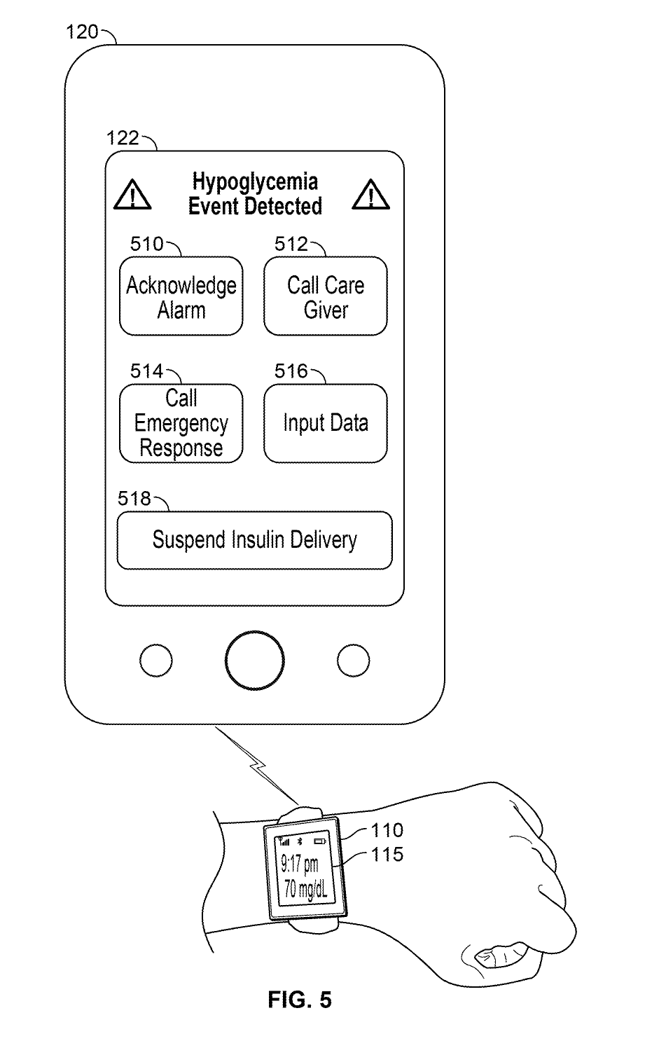

Depending on the configuration of the system 100, the alarm condition may optionally be wirelessly communicated from the wearable physiological sensing device 110 to one or more of the external devices 120, 130, 140, 150, 160, 170, 180 (via the network 125), and 190 (via the network 125) of the system 100. For example, in some embodiments the alarm condition may be communicated to the user's computing device 120, the base station 130, the alarm clock 140, or combinations thereof. In some embodiments, the user of the wearable physiological sensing device 110 may acknowledge and silence the alarm via the user interface (e.g., using a touchscreen, one or more depressible buttons, a microphone, or a combination thereof) of the user's computing device 120, the base station 130, or the alarm clock 140. As will be described further below in reference to FIG. 5, in particular embodiments the user may also initiate other actions via the user's computing device 120. In another example, the alarm condition may be wirelessly communicated from the sensing device 110 to a vehicle equipped with Bluetooth communication equipment or other wireless communication equipment (e.g., so as to output an alarm to from sensing device 110 to the vehicle's audio system or other equipment therein while the user is in the vehicle).

In some embodiments of the system 100, the detected alarm event can be further communicated to other external devices in the system 100. For example, a caregiver's computing device 180 may receive a communication corresponding to the alarm (even when the caregiver is in remote location, such as in a different building, city or state from the user of the sensing device 110). The communication may be initiated from any of the aforementioned devices such as the user's computing device 120, the base station 130, the alarm clock 140, the wearable physiological sensing device 110, and combinations thereof. The alarm may be communicated using various communication modes such as, but not limited to, a cellular phone network, Wi-Fi, a computer network 125 (e.g., the internet), land-based telephony systems, and the like, and combinations thereof. The communication may be output to the caregiver in the form of a telephone call, a SMS text message, an email, activation of an alarm of an application that has been installed at the caregiver's computing device 180, and the like.

A remote monitoring station 190 may also receive a communication relating to the detected alarm event in some embodiments of the system 100. Such communication can be initiated and transmitted using the same techniques as described in relation to the caregiver's computing device 180. The remote monitoring station 190 may be a centralized telemetric monitoring system (located remotely from the user of the sensing device 110, such as in a different building, city or state) used by a health care provider such as a clinic, hospital, research facility, and the like. The data stored by the remote monitoring station 190 may be useful for defining or refining a treatment plan for the user of the wearable physiological sensing device 110, monitoring the effectiveness of a treatment plan, determining the user's compliance with a treatment plan, and the like. The remote monitoring station 190 includes a data repository that can be located at the health care provider's facility, as part of a computer network of the health care provider, or the data repository can be stored on a network accessible by one or more individual health care providers associated with the particular user of the sensing device 110 (e.g., a cloud-based data repository accessible via the Internet using a private password or other secure login), in particular implementations. In some embodiments, a clinician can access the remote monitoring station 190 via a computer network and then initiate an uploading of new or modified configuration settings into the wearable physiological sensing device 110.

In some embodiments of the system 100, or based upon the particular circumstances associated with the detected alarm event at the sensing device 110, a communication corresponding to the alarm may be sent to an emergency call center 195. The emergency call center 195 can be a remote emergency monitoring and dispatch service, a governmental “9-1-1” call center, and the like. These communications can be sent via modalities such as a cellular telephone network, land-based telephone network, internet, and other communication modes and combinations thereof. The communications can originate, for example, from the wearable physiological sensing device 110, the user's computing device 120, the base station 130, or the alarm clock 140. In some embodiments, the communications can be a two-way voice communication, a textual message that corresponds to the user, an automated voice message, or another type of communication that alerts the emergency call center 195 to the alarm condition.

With reference to FIG. 2, in some embodiments the wearable physiological sensing device 110 is configured physically similar to a form factor of a wristwatch, such as the sensing device 110 that includes a wearable housing 112 and a band 114. In other embodiments, other form factors of the wearable physiological sensing device are used (e.g., an adhesive patch, an armband, a headband, a belt or strap mounted sensing device, a device that is integrated with clothing articles, a clip-on configuration, and the like). The band 114 is adjustable in size to fit a range of users and to fit various body parts (e.g., wrist or ankle). In this embodiment, the band has two flexible straps that are coupled together using a clasp device 115. In other embodiments, other types of strap closures can be used (e.g., hook and loop materials, snaps, magnets, and the like) or an elastic band, malleable strap members, and the like, can be substituted for the flexible straps.

In this embodiment, the housing 112 of the sensing device 110 contains multiple modules, devices, circuits, and subsystems that function cooperatively to perform the operations of the physiological sensing device 110 as described herein.

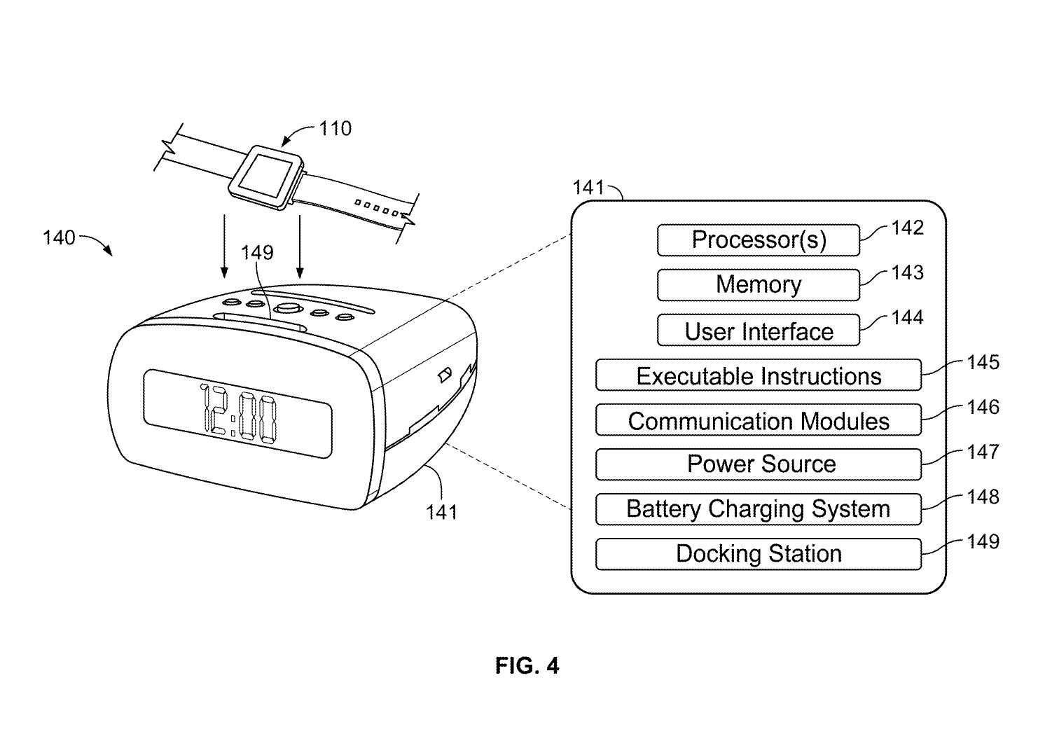

For example, a power source 208 is located within the housing 112. The power source 208 can provide the energy to operate the other devices and systems of the physiological sensing device 110. In some embodiments, the power source 208 comprises one or more batteries such as a non-rechargeable alkaline battery. In some embodiments, the power source 208 is one or more rechargeable batteries such as a nickel-metal hydride, lithium ion, lithium polymer, or zinc oxide battery. In particular embodiments, a combination of rechargeable and non-rechargeable batteries are used. The rechargeable batteries may be recharged by electrically coupling an external power source to the battery, or to a battery charging circuit in the housing 112 that is electrically connected to the power source 208. In some embodiments, the coupling of the external power source to the sensing device 110 is via a wired connection, such as by plugging a cord into a receptacle located on the housing 112. The coupling may also be accomplished in some embodiments by the use of a docking station (e.g., refer to FIG. 4) with which the physiological sensing device 110 can mate to establish an electrical connection. In particular embodiments, the electrical coupling can be accomplished inductively (wirelessly). That is, an electrical coil that is within the housing 112 can be wired to a battery charging circuit in the housing 112. The internal electrical coil can receive inductive energy via an alternating magnetic field emanating from a primary coil that is part of an external charging station. An alternating current is thereby induced in and transmitted from the internal coil to the battery charging circuit in the housing 112. The battery charging circuit can rectify the alternating current to produce direct current that is used to charge the power source 208.

The housing 112 can also contain modules, devices, control circuitry, and subsystems such that the physiological sensing device 110 constitutes a mobile computing device. As such, the housing 112 may contain the components and subsystems of a mobile computing device including, but not limited to, one or more processors 210, computer-readable memory devices 212 containing executable instructions 214, a user interface 216, and communication modules 218 (e.g., including one or more wireless communication devices, as previously described herein).

In some embodiments, the physiological sensing device 110 is configured to operate as a smartwatch, in addition to performing the physiological monitoring and alarming functions described herein. In some such embodiments, the sensing device 110 may operate in conjunction with another computing device such as the user's computing device 120 of FIG. 1. Such smartwatch functions can include, but are not limited to, provision of notifications (e.g., notifications corresponding to the receipt of SMS messages, e-mail messages, telephone calls, calendar events, and the like), control of smart phone functions (e.g., playing of music, use of applications, and so on). Such functions can be presented and controlled through the user interface 216.

The control circuitry housed in the sensing device 110 may be implemented a combination of processor(s) 210, the computer-readable memory 212 (which may optionally store executable instructions 214 configured to perform the sensing and determination operations described herein). The processor(s) 210 are suitable for the execution of one or more computer programs and include, by way of example, both general and special purpose microprocessors, and any one or more processors of any kind of digital computer. The processor(s) 210 can execute instructions, including the executable instructions 214 that are stored in the memory 212. The processor(s) 210 may be implemented as a chipset of chips that include separate and multiple analog and digital processors. The processor(s) 210 may provide, for example, for coordination of the other components of the physiological sensing device 110, such as control of the user interface 216, applications run by the physiological sensing device 110, and wireless communications via the communication modules 218.

The computer-readable memory 212 stores information within the physiological sensing device 110, including, but not limited to, the executable instructions 214. The memory 212 can be implemented as one or more of a computer-readable medium or media, a volatile memory unit or units, or a non-volatile memory unit or units. An expansion memory may also be provided and connected to the physiological sensing device 110 which may include, for example, a SIMM (Single In-Line Memory Module) card interface. The expansion memory may provide extra storage space for the physiological sensing device 110, or may also store applications or other information for the physiological sensing device 110. The memory 212 may include, for example, flash memory and/or NVRAM memory (non-volatile random access memory).

The executable instructions 214 can be stored in the memory 212, the expansion memory, memory on the processor 210, or in a combination thereof. The executable instructions 214 can include instructions that, when executed, perform functions related to the operating systems of the physiological sensing device 110 (e.g., operations of the user interface 216, coordination of intra-device module communications, coordination and control of applications run by the sensing device 110, and so on). In addition, in this embodiment the executable instructions 214 include instructions that, when executed, perform one or more of the functions and methods described elsewhere herein in relation to physiological parameter monitoring, analysis of the monitored parametric data, alarming, and communications with other devices and systems. In some implementations, the executable instructions 214, or portions thereof, can be received in a propagated signal, for example, via the communication modules 218.

To provide for interactions with a user, the physiological sensing device 110 can also include a user interface 216. The user interface 216 includes devices and systems to receive inputs to sensing device 110, and to provide outputs from the sensing device 110. For example, in some embodiments the user interface 216 can include a display 115 (in some embodiments the display 115 is a touchscreen display), one or more buttons 116 that can be soft keys or hard keys, one or more audio speakers, one or more lights, a microphone, a camera, tactile feedback mechanisms (e.g., vibratory alarm signals), and the like. Using such devices, the user interface 216 can receive user input including voice input, touchscreen input, soft key inputs, and the like. Additionally, some devices of the user interface 216 (such as the buttons 116) can receive user input indicating that other devices of the user interface 216 should be activated; for example, in some embodiments in which one or more buttons 116 are actuated by the user so as to activate the internal microphone and audio speaker and furthermore initiate an emergency verbal communication line (via the network 125 or cellular communication) with the caregiver device 180, the remote monitoring station 190 or the emergency call center 195. The user interface 216 can also provide outputs including audible alarms or messages, visual alarms or messages, tactile alarms or messages, differentiation of alarm types, and the like.

The physiological sensing device 110 may communicate wirelessly with one or more of the external devices 120, 130, 140, 150, 160, 170, 180 (via the network 125), and 190 (via the network 125) through the communication interface modules 218, which may include digital signal processing circuitry where necessary. The communication modules 218 may provide for communications using various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through a transceiver using a radio-frequency. In addition, short-range communication may occur via communication interface modules 218, such as by using Bluetooth, WiFi, RFID, ANT+, NFC, or other such transceivers (not shown). In addition, a GPS (Global Positioning System) receiver module 220 may provide additional navigation and location-related wireless data to the physiological sensing device 110, which may be used as appropriate by applications running on the physiological sensing device 110.

The physiological sensing device 110 can also include a perspiration sensor 220. The perspiration sensor 220 can be used, for example, for detecting an increase in perspiration that can signify a hypoglycemic condition. In particular embodiments, the increase in perspiration can be detected as distinct from variations in basal perspiration that is from physiological behaviors that are unrelated to blood glucose concentration.

In some embodiments, the perspiration sensor 220 measures the galvanic skin resistance (GSR) of the user of the sensing device 110. GSR refers to the measured electrical resistance between two electrodes when a very weak current is steadily or periodically passed between them. Accordingly, some embodiments of the perspiration sensor 220 include two electrodes that are in contact with the skin of the user. For example, the two electrodes can be mounted to the housing 112 and at least partially exposed along the exterior rear surface 113 (FIG. 2) of the housing 112 and spaced apart from each other. The perspiration sensor 220 can periodically measure the resistance between the two electrodes. The measurement, and/or a trending of multiple measurements over time, can be used as an input to an algorithm for detecting a health event, such as a hypoglycemic event. Thus, the perspiration sensor 220 can be an electrical resistance-type moisture sensor that utilizes the relationship between the amount of moisture on the skin and the electrical resistance of the skin. In particular, perspiration sensor 220 can operate on the principle that skin's resistance to the flow of electricity is lessened with increasing amounts of moisture such as perspiration. When the skin's GSR is lessened by perspiration, it can more readily conduct electricity and the flow of electricity can be detected by a monitoring circuit of the perspiration sensor 220. Using these principles, the perspiration sensor 220 can be used to detect the presence of perspiration that may be determined to be at or above a threshold level amount of perspiration that is indicative of a health event, such as hypoglycemia.

The physiological sensing device 110 can also include a skin temperature sensor 222. The skin temperature sensor 222 can be used, for example, for detecting a decrease in skin surface temperature that can signify a hypoglycemic condition. In some embodiments, the skin temperature sensor 222 can include a thermistor that is used to detect the skin surface temperature, and the thermistor can be mounted to the housing 112 and at least partially exposed along the exterior rear surface 113 (FIG. 2) of the housing 112. A thermistor can be a type of resistor whose resistance varies significantly with temperature, more so than in standard resistors. In some embodiments, the thermistor is within a bridge circuit of the skin temperature sensor 222. The skin temperature sensor 222 can periodically measure the skin surface temperature. The measured skin temperature value, and/or trends over time of such values, can be used to detect a change in skin temperature that may be determined to be at or above a threshold level that is indicative of a health event, such as hypoglycemia. In addition, the skin temperature sensor 222 can be used to confirm that the user is wearing the physiological sensing device 110. That is, in some embodiments the physiological sensing device 110 can be configured to send a corresponding message to a caregiver or medical personnel when the skin temperature sensor 222 is below a minimum temperature value for over a threshold period of time.

The physiological sensing device 110 can also optionally include a GPS device 224 mounted within the housing 112. In some embodiments, the GPS device 224 is a GPS transceiver that can send and receive microwave transmissions to and from GPS satellites to ascertain the location of the sensing device 110. GPS transceivers are composed of an antenna that is tuned to the frequencies transmitted by the GPS satellites, receiver-processors, and a clock (such as a crystal oscillator). In particular embodiments, the GPS device 224 is an active GPS tracking system. An active GPS tracking system automatically sends the information from the GPS transceiver to a central tracking portal or system in real-time as it happens. The active GPS tracking system can be used to track or monitor the location of the user of the sensing device 110. Accordingly, in such embodiments the GPS device 224 can allow a caregiver or medical personnel to know the geographic location of the user of the physiological sensing device 110, which may be helpful in the event of an emergency (e.g., when a detected alarm event identified by the sensing device 110 is not acknowledged or remedied by the user, or another medical emergency event).

Still referring to FIG. 2, the physiological sensing device 110 can also optionally include a pedometer 226 mounted within the housing 112. The pedometer 226 can be used to measure the physical activity of the user of the sending device 110. In some embodiments, the pedometer 226 includes one or more accelerators that are used to detect motion of the sensing device 110. The detected motion can be correlated to calories burned by the user wearing the sensing device 110. In particular embodiments, the calories burned can be used as a factor for the calculation of an amount of insulin to be delivered to the user (e.g., such as a bolus or basal insulin delivery by an insulin pump). The pedometer 226 can also provide an input to the algorithms for detecting hypoglycemia events in some embodiments. For example, if the perspiration sensor 220 detects an increase in perspiration, the algorithm for determining hypoglycemia may attribute the perspiration increase to physical activity by the user if the pedometer 226 indicates such activity. Also, the data collected by the pedometer 226 can be wireless communicated to other external devices (including the caregiver's computing device 180 and the remote monitoring station 190) so that the caregiver and the healthcare provider can receive updates regarding the user's activity (e.g., periodic remote monitoring of the user's exercise or activity levels).

The physiological sensing device 110 can also optionally include one or more accelerometers 228 mounted within the housing 112, and/or other types of motion sensors such as gyroscopes. The accelerometers 228 are an electronic component that measures tilt and motion. The accelerometers 228 are also capable of detecting rotation and motion gestures such as swinging or shaking. In some embodiments, the accelerometers 228 can be configured to detect gestures for particular input commands, such as the acknowledgement of an alarm and other types of commands. The accelerometers 228 can also be used to detect a lack of motion, which can then be wireless communicated to other external devices (including the caregiver's computing device 180 and the remote monitoring station 190). This feature can be used, for example, to provide additional information to a caregiver or medical personnel when the sensing device 110 is alarming. For example, in such situations a lack of motion by the user may indicate that the user is in a coma, or is otherwise incapacitated.

The physiological sensing device 110 can also optionally include an oximetry system 230. The oximetry system 230 can be used to detect health parameters such as the user's pulse rate and the oxygen saturation of the blood of the user. Oximetry systems operate on the basis that the transmission and absorption of near infrared light in human body tissues contains information about hemoglobin concentration changes. In some embodiments, the oximetry system 230 can be configured as a regional oxygen saturation (rSO2) system. Such oximetry systems 230 utilize near infrared light which is emitted from a light source (e.g., a LED) at the user's skin surface, then penetrates the user's epidermis, and returns to the skin's surface to be sensed by a detector. In some such embodiments, the light source and detector both can be mounted to the housing 112 and at least partially exposed along the exterior rear surface 113 (FIG. 2) of the housing 112 so as to contact with the skin 102 of the user. The data collected by the oximetry system 230 can be used in on-board algorithms and/or telemetrically transmitted to other devices, such as computing devices of the user's caregiver or medical personnel.

Still referring to FIG. 2, the physiological sensing device 110 can also optionally include a graphene-based glucose sensor 232. In some embodiments, graphene can be coated on a metal wire (e.g., platinum) to create an electrochemical sensor that is responsive to the glucose concentration in the user's blood. The graphene-coated wire can be configured as an electrode that can penetrate the user's skin to be in contact with the user's blood. In some embodiments, the graphene-based glucose sensing electrode can be mounted to the housing 112 and at least partially extend outwardly from the exterior rear surface 113 (FIG. 2) of the housing 112. In alternative embodiments, the graphene-based glucose sensing electrode can be separately attached to the user's skin and spaced apart from the housing 112 and in communication with the physiological sensing device 110 by a wired or a wireless connection.

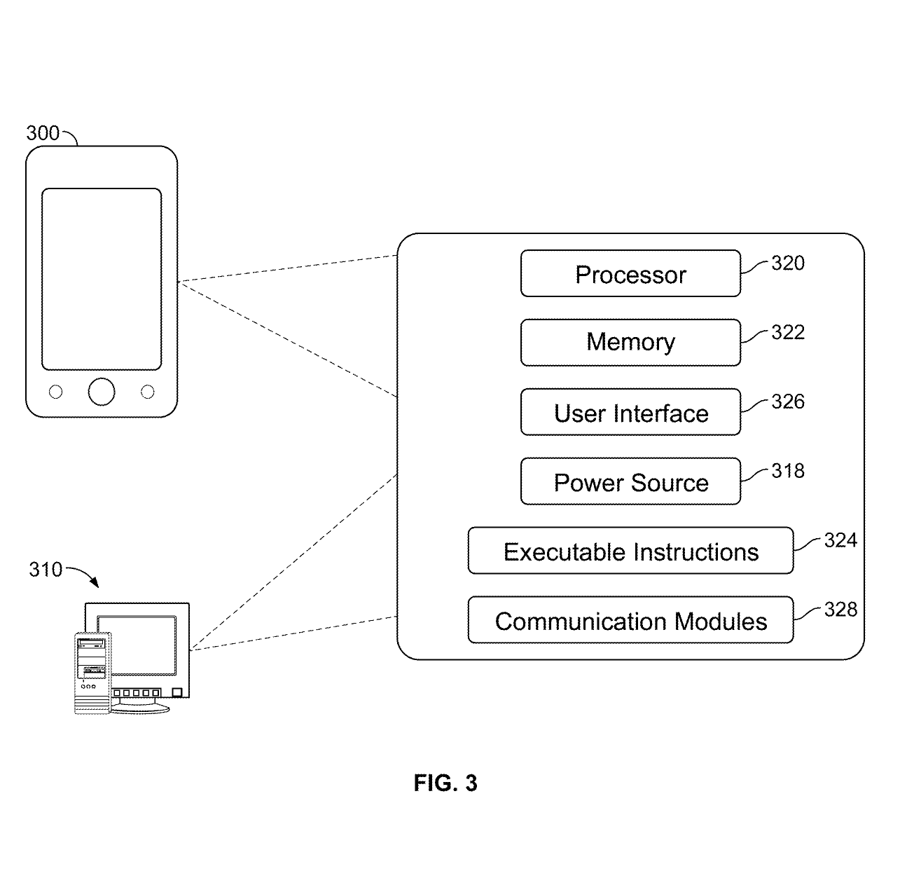

Referring now to FIG. 3, a mobile computing device 300 and a desktop computing device 310 are included in some embodiments of the system 100 system. For example, the mobile computing device 300 can be implemented as one or more of the user's computing device 120, the caregiver's computing device 180, or another external computing device of a physician or other healthcare provider. In another example, the desktop computing device 310 can be implemented as one or more of the user's computing device 120, the caregiver's computing device 180, the remote monitoring station 190, a workstation of the emergency call center 195, or another external computing device of a physician or other healthcare provider. The mobile computing device 300 and the desktop computing device 310 can be used within the system 100 to perform functions including, but not limited to, receiving physiological data or detected alarm conditions from the sensing device 110, determining alarm status conditions, sending data, recording data, and the like.

The mobile computing device 300 can be any of a number of different types of mobile computing devices, such as a smartphone, a tablet PC, a laptop PC, a PDA, a wearable computer, and the like. The mobile computing device 300 can be conveniently used as a computing device by the user of the wearable physiological sensing device (e.g., user's computing device 120 of FIG. 1). In addition, the mobile computing device 300 can be conveniently used as a computing device for a caregiver (e.g., caregiver's computing device 180 of FIG. 1). However, other types of computing devices (e.g., a desktop PC) can be substituted for the mobile computing device 300. In this example, the mobile computing device 300 is a smartphone.

The desktop computing device 310 can be included in some embodiments of a physiological sensing system. For example, the remote monitoring station 190 of example physiological sensing system 100 (refer to FIG. 1) is depicted as a desktop computing device 310.

The mobile computing device 300 and the desktop computing device 310 can include multiple modules, devices, and systems that function cooperatively to perform operations of computing devices as part of the systems provided herein. The following description of the modules, devices, and systems of the mobile computing device 300 and the desktop computing device 310 is also applicable to a computing device that is implemented as a base station, such as the base station 130 of FIG. 1.

For example, the mobile computing device 300 and the desktop computing device 310 include a power source 318. The power source 318 can provide the energy to operate the other devices and systems of the mobile computing device 300 and the desktop computing device 310. In some embodiments of the mobile computing device 300, the power source 318 is one or more rechargeable batteries such as a nickel-metal hydride, lithium ion, lithium polymer, or zinc oxide battery. The rechargeable batteries may be recharged by electrically coupling an external power source to the battery, or to a battery charging circuit in the mobile computing device 300 that is electrically connected to the power source 318. In some embodiments, the coupling of the external power source to the mobile computing device 300 is via a wired connection, such as by plugging a cord into a receptacle located on the mobile computing device 300. The coupling may also be accomplished in some embodiments by the use of a docking station with which the mobile computing device 300 can mate to establish an electrical connection. In particular embodiments, the electrical coupling can be accomplished inductively as described above in reference to the physiological sensing device 110. In some embodiments of the desktop computing device 310, the power source 318 is supplied with AC from a wall outlet and the power source 318 converts the AC to DC power to supply the components and systems of the desktop computing device 310.

The mobile computing device 300 and the desktop computing device 310 can include one or more processor 320. The processor(s) 320 are suitable for the execution of a computer program and can be, by way of example, both general and special purpose microprocessors, and any one or more processors of any kind of digital computer. The processor(s) 320 can execute instructions, including the executable instructions 324 that are stored in the memory 322. The processor(s) 320 may be implemented as a chipset of chips that include separate and multiple analog and digital processors. The processor(s) 320 may provide, for example, for coordination of the other components of the mobile computing device 300 and the desktop computing device 310, such as control of the user interface 326, applications run by the mobile computing device 300 and the desktop computing device 310, and communications via the communication modules 328.

The memory 322 stores information within the mobile computing device 300 and the desktop computing device 310, including, but not limited to, the executable instructions 324. The memory 322 can be implemented as one or more of a computer-readable medium or media, a volatile memory unit or units, or a non-volatile memory unit or units. An expansion memory may also be provided and connected to the mobile computing device 300 and the desktop computing device 310, that may include, for example, a SIMM (Single In-Line Memory Module) card interface. The expansion memory may provide extra storage space for the mobile computing device 300 and the desktop computing device 310, or may also store applications or other information for the memory. The memory 322 may include, for example, flash memory and/or NVRAM memory (non-volatile random access memory).

The executable instructions 324 can be stored in the memory 322, the expansion memory, memory on the processor(s) 320, or in a combination thereof. The executable instructions 324 can include instructions that, when executed, perform functions related to the operating systems of the mobile computing device 300 or the desktop computing device 310 (e.g., operations of the user interface 326, coordination of intra-device module communications, coordination and control of applications run by the mobile computing device 300 and the desktop computing device 310, and so on). In addition, in this embodiment the executable instructions 324 include instructions that, when executed, perform one or more of the functions and methods described elsewhere herein in relation to physiological parameter monitoring, analysis of the monitored parametric data, alarming, and communications with other devices and systems. In some implementations, the executable instructions 324, or portions thereof, can be received in a propagated signal, for example, via the communication modules 328.

Still referring to FIG. 3, to provide for interactions with users, the mobile computing device 300 and the desktop computing device 310 can also include a user interface 326. The user interface 326 includes devices and systems to receive inputs to mobile computing device 300 and the desktop computing device 310, and to provide outputs from the mobile computing device 300 and the desktop computing device 310. For example, in some embodiments the user interface 326 can include a display (in some embodiments the display is a touchscreen display), one or more buttons that can be soft keys or hard keys, a keyboard, a mouse, one or more acoustic speakers, one or more indicator lights, a microphone, a camera, tactile feedback mechanisms (e.g., vibratory alarm signals), and the like. Using such devices, the mobile computing device 300 and the desktop computing device 310 can receive user input including voice input, touchscreen input, mouse input, keyboard input, soft key input, and the like. The user interface 326 can also provide outputs including visual alarms or messages, audible alarms or messages, tactile alarms or messages, differentiation of alarm types, and the like.

The mobile computing device 300 and the desktop computing device 310 may communicate through the communication interface modules 328, which may include digital signal processing circuitry where necessary. The communications may be performed wirelessly, or through wired network connections. The communication modules 328 may provide for communications using various modes or protocols, such as GSM voice calls, SMS messaging, EMS messaging, MMS messaging, CDMA, TDMA, PDC, WCDMA, CDMA2000, or GPRS, among others. Such communication may occur, for example, through a transceiver using a radio-frequency. In addition, short-range communication may occur via communication interface modules 328, such as by using Bluetooth, WiFi, RFID, ANT+, NFC, or other such transceivers (not shown). In addition, in some embodiments of the mobile computing device 300 a GPS receiver module (not shown) can be included. The GPS receiver may provide additional navigation and location-related wireless data which may be used as appropriate by applications running on the mobile computing device 300.

Referring now to FIG. 4, the alarm clock 140 is included in some embodiments of a physiological sensing and response system, such as the example system 100 of FIG. 1. The alarm clock 140 can be used within a physiological sensing system to perform functions including, but not limited to, receiving physiological data, determining alarm status conditions, providing alarms or messages, sending data, recording data, charging a wearable physiological sensing device, and the like.

The alarm clock 140 can include a housing 141, one or more processors 142, memory 143, user interface 144, executable instructions 145, communication modules 146, power source 147, battery charging system 148, and docking station 149. The processor(s) 142, memory 143, executable instructions 145, communication modules 146, and power source 147 can be analogous to the corresponding components and systems of the mobile computing device 300 and the desktop computing device 310 described above.

The user interface 144 of the alarm clock 140 can include the typical functions of an alarm clock, such as displaying the time of day, alarming based on a time set-point, and so on. In addition, the user interface 144 can include devices that are responsive to messages or alarms from a wearable physiological sensing device such as the wearable physiological sensing device 110. For example, the user interface 144 can sound audible alarms or messages and/or present visual alarms or messages resulting from measured physiological parameters of the user that exceed threshold limit values. Such measured physiological parameters can include, but are not limited to, skin temperature, perspiration, oxygen levels in the blood, pulse rate, movement, and blood glucose levels. The user interface 144 can also include user input devices, such as buttons, by which the user can acknowledge and silence messages or alarms, activate/deactivate the alarm functionality, configure types of alarms to be used, and the like.

In some embodiments, the alarm clock 140 can also include a docking station 149 that is built into the housing 141. The docking station 149 can include exterior mounting structures configured to couple with a wearable physiological sensing device, such as the wearable physiological sensing device 110. In some embodiments, the docking station 149 includes an electrical connector that couples with a complementary electrical connector located on the wearable physiological sensing device 110. For example, the electrical connector of the docking station 149 can be a male connector and a corresponding female connector can be located on the sensing device 110. The electrical connection between the alarm clock 140 and the docking station 149 can be used to transmit electrical energy to the wearable physiological sensing device 110. The electrical energy can power the operations of the wearable physiological sensing device 110, can recharge the on-board battery or batteries of the sensing device 110, or a combination thereof.

In some embodiments, the alarm clock 140 can charge the on-board battery or batteries of the wearable physiological sensing device 110 inductively (wirelessly). A primary coil that located within the housing 141 can receive AC from the battery charging system 148. When the wearable physiological sensing device 110 is physically close to the alarm clock 140 (such as within the docking station 149) a secondary electrical coil within the wearable physiological sensing device 110 can receive inductive energy via an alternating magnetic field emanating from primary coil of the alarm clock 140. Alternating current is thereby induced in and transmitted from the internal coil of the wearable physiological sensing device 110 to a battery charging circuit in the sensing device 110. The battery charging circuit can rectify the alternating current to produce direct current that can be used to charge the battery or batteries of the wearable physiological sensing device 110.

The alarm clock device 140 can wirelessly communicate with the wearable physiological sensing device 110 while the sensing device 110 is worn on the body of the user (for example, while the user is sleeping in a nearby bed). For example, in response to the sensing device 110 detecting health parameters indicative of a health condition, such as a hypoglycemia event, the alarm clock 140 can wirelessly receive such alarm condition information from the sensing device and then automatically response by outputting an alarm to wake the user. Many hypoglycemia events can occur at night (due to a low blood-glucose level of the user that is sleeping), and the user's health may further deteriorate during the sleeping state if remedial actions are not promptly performed by the user or a caretaker. As such, the system 100 can be particularly useful when implemented by a user during the normal sleeping hours so as to reduce the likelihood of future hypoglycemia events that might otherwise go undetected and dangerously untreated.

Referring to FIG. 5, some embodiments of the system can employ a combination of the wearable physiological sensing device 110 and the user's computing device 120. The user's computer device 120 can wirelessly communicate with the wearable physiological sensing device 110 so as to respond to an alarm condition detected by the sensing device 110 (while the sensing device 110 is worn on the body of the user). In this example depicted in FIG. 5, the sensing device 110 detects a hypoglycemia alarm event, but it should be understood from the description herein that other types of alarms or messages relating to other health parameters can operate similarly.

In this example, a hypoglycemia event alarm can be transmitted by the sensing device 110 to the user's computing device 120 upon a determination by the physiological sensing device 110 that the symptoms of hypoglycemia have been detected. Accordingly, an alarm, such as an audible and/or visual alarm, can be emitted at the physiological sensing device 110 as well as at the user's computing device 120. In some embodiments, such alarms are selectable and configurable in accordance with user preferences as will be described further below. In particular embodiments, the severity of the detected physiological condition can be indicated by the color of the alarm displayed (e.g., yellow for mild conditions and red for severe conditions), and/or by the tone of the alarm emitted, and the like. In response to the alarm, the user may interact with the user interface 115 of the wearable sensing device 110. In addition or in the alternative, the user may respond to the alarm by interacting with the user's computing device 120, as depicted by this example.

In this example, a touchscreen display 122 of the user's computing device 120 presents to the user various options for responding to the hypoglycemia event alarm. The user may make one, or in some cases more than one, selection of the options displayed on the touchscreen display 122. For example, if the user wants the hypoglycemia event alarm to be silenced, the user can select “Acknowledge Alarm” 510. The user may choose to make such a selection if, for example, the user has taken remedial actions in response to the hypoglycemia event alarm, such as by consuming fast-acting carbohydrates, glucose tablets, or candy, and discontinuing insulin intake. The user may also choose to make such a selection if, for example, the user has deemed the hypoglycemia event alarm to be a false alarm. After selecting “Acknowledge Alarm” 510, the alarm is silenced and the touchscreen display 122 may continue to display the response options as shown, at least for a predefined period of time. For example, the user can select the touchscreen button 510 so as to silence or “snooze” the alarm for a period of about 1 minute to about 15 minutes, and preferably about 5 minutes, to thereby provide the user with a limited period of time to take corrective actions (e.g., to ingest fast-acting carbohydrates, glucose tablets, or candy, to discontinue any insulin dosages, to a combination thereof).

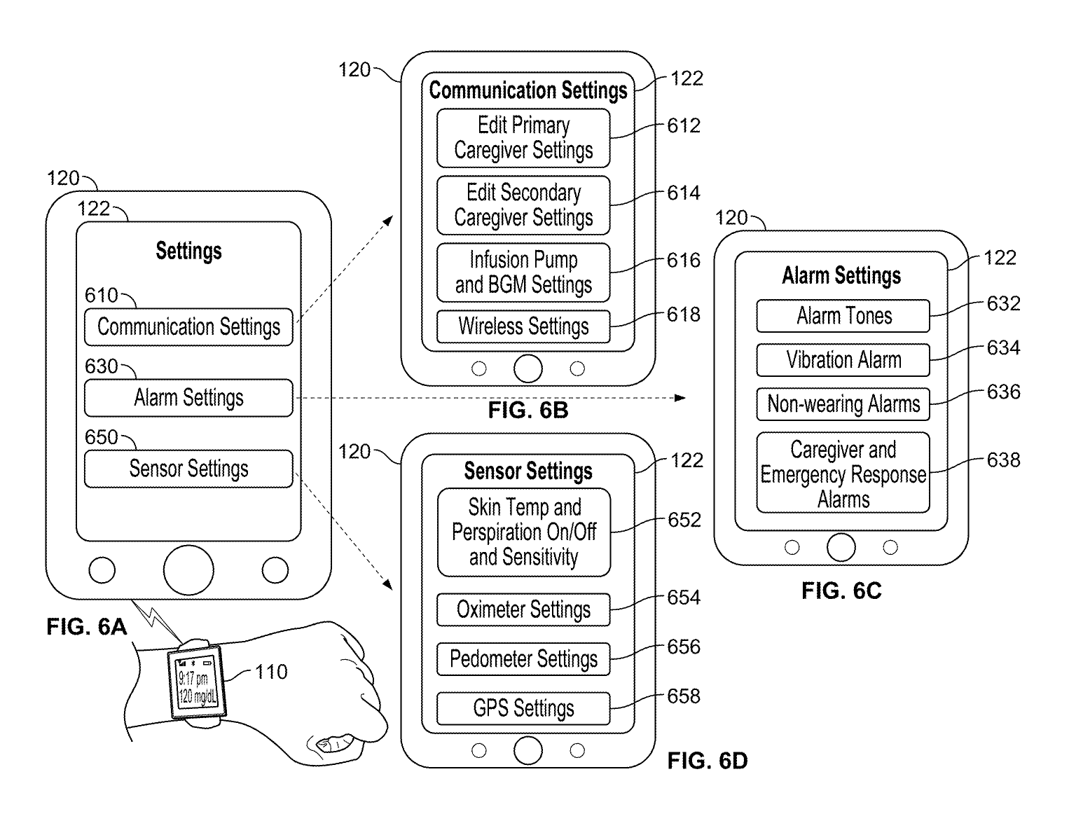

The user can make a selection of “Call Caregiver” 512 to initiate a telephone call to the user's designated caregiver (as configured by the user, as described below in reference to FIG. 6B). The user may choose to make such a selection if, for example, the user needs the assistance of the caregiver. The user may also choose to make such a selection if, for example, the user want to inform the caregiver about circumstances regarding the alarm, such as the remedial actions that the user has taken. For whatever reason, the user can select “Call Caregiver” 512 to initiate a telephone call to the caregiver.

The user can make a selection of “Call Emergency Response” 514 to initiate a telephone call to an emergency call center. The user may choose to make such a selection if, for example, the user decides that emergency medical assistance is needed, such as from an emergency medical technician (EMT) and/or an ambulance service.

Still referring to FIG. 5, the user can make a selection of “Input Data” 516 to provide information that will be stored for future analysis. The user may choose to make such a selection if, for example, the user wants to provide some contextual information relating to the hypoglycemia event alarm. In some embodiments, upon the receipt of the selection of “Input Data” 516, the touchscreen display 122 may provide a soft-keyboard for the user to enter the information. In particular embodiments, the user's computing device 120 may facilitate voice entry of information from the user. Some examples of the kinds of information that the user might choose to provide could include, “I performed 30 minutes of heavy exercise,” “I skipped a meal,” “I took too much insulin,” “false alarm,” and the like. The information can be stored in memory along with the physiological data relating to the hypoglycemia event alarm. The memory that stores such data and information may be in the wearable sensing device 110, the user's computing device 120, at a remote monitoring station (e.g., remote monitoring station 190 of FIG. 1), or a combination thereof. The contextual information may be helpful for defining or refining a treatment plan for the user of the wearable physiological sensing device 110, monitoring the effectiveness of a treatment plan, determining the user's compliance with a treatment plan, and the like.

In some embodiments, the user can make a selection of “Suspend Insulin Delivery” 518 in response to a hypoglycemia event alarm. As described above, particular embodiments of a physiological sensing system, such as the example physiological sensing system 100 of FIG. 1, include an infusion pump system in communication with the wearable physiological sensing device 110 and/or the user's computing device 120. In some such embodiments, in response to a hypoglycemia event alarm, the user may choose to select “Suspend Insulin Delivery” 518. In response to a user selection of “Suspend Insulin Delivery” 518, a command is communicated from the wearable physiological sensing device 110 and/or the user's computing device 120 to the infusion pump system to stop insulin dispensations. Thereafter, the wearable physiological sensing device 110 and/or the user's computing device 120 may periodically alert the user to the fact that infusion pump system dispensations of insulin have been suspended. Alternatively, the selection of “Suspend Insulin Delivery” 518 may result in a temporary suspension of insulin dispensations from the infusion pump system, such as for a period of 30 minutes, 1 hour, 1.5 hours, 2 hours, 2.5 hours, 3 hours, 3.5 hours, 4 hours, or more than 4 hours. Thereafter, the wearable physiological sensing device 110 and/or the user's computing device 120 may send a command to reactivate the infusion pump system.

Referring now to FIGS. 6A through 6D, in some embodiments of the system 100, the user's computer device 120 in communication with the wearable physiological sensing device 110 can be used to configure the settings of the system 100 system. It should be understood from the description herein that FIGS. 6A through 6D provide some non-limiting examples of the types of settings that can be configured, and these examples may not be exhaustive. In addition, the selection of some types of settings may result in the presentation on the display 122 of one or more additional screen configurations by which the user can enter or adjust the settings. It should be understood from the description herein that such other types of settings and additional screen configurations are within the scope of this disclosure.

Turning now to FIG. 6A, the user's computer device 120 in communication with the wearable physiological sensing device 110 can be used to configure the settings of a physiological sensing system, such as “Communication Settings” 610, “Alarm Settings” 630, and “Sensor Settings” 650. While this example shows the wearable physiological sensing device 110 on the wrist of the user, there is no requirement for the wearable physiological sensing device 110 to be on the wrist of the user at the time that the settings are accessed and selected.

When a selection of “Communication Settings” 610 is made, in some embodiments the configuration of FIG. 6B is presented on the touchscreen display 122 of the user's computing device 120. The selections presented in the category of “Communication Settings” can include, but are not limited to, “Edit Primary Caregiver Settings” 612, “Edit Secondary Caregiver Settings” 614, “Infusion Pump and BGM Settings” 616, and “Wireless Settings” 618.

Referring now to FIG. 6B, the user can select “Edit Primary Caregiver Settings” 612 to input contact information for the user's primary caregiver. Such contact information may include the primary caregiver's landline telephone number, cellular telephone number, email address, pager number, and the like. The primary caregiver's contact information can be used, for example, for sending a notification to the primary caregiver when an alarm condition for the wearable physiological sensing device 110 exists. Such notifications may be sent in the form of an email, a SMS text message, an alarm notification for a physiological sensing system application running on the primary caregiver's computing device, a telephone call, and the like. Another example use for the primary caregiver's contact information is when the user selects “Call Caregiver” 512 (refer to FIG. 5). In such a case, a phone call can be initiated from the user's computing device 120 to the primary caregiver's designated telephone number.

The user can select “Edit Secondary Caregiver Settings” 614 to input contact information for the user's secondary caregiver. Such contact information may include the secondary caregiver's landline telephone number, cellular telephone number, email address, pager number, and the like. The secondary caregiver's contact information can be used, for example, for sending a notification to the secondary caregiver when an alarm condition for the wearable physiological sensing device 110 exists. In other words, in some cases the user of the wearable physiological sensing device 110 may want notifications sent to both the primary and secondary caregivers. Such notifications may be sent in the form of an email, a SMS text message, an alarm notification for a physiological sensing system application running on the caregiver's computing device, a telephone call, and the like. Another example use for the secondary caregiver's contact information is when the user selects “Call Caregiver” 512 (refer to FIG. 5). In such a case, if a phone call initiated from the user's computing device 120 to the primary caregiver's designated telephone number is unanswered, a telephone call to the secondary caregiver's designated telephone number may be automatically initiated from the user's computing device 120, when the system is so configured.

Still referring to FIG. 6B, another setting the user can select from the “Communication Settings” category is “Infusion Pump and BGM Settings” 616. The “Infusion Pump and BGM Settings” 616 can be used to configure communications when the user's physiological sensing system includes an infusion pump and/or a BGM, such as the example physiological sensing system 100 of FIG. 1 that includes the BGM device 160 and the portable infusion pump system 170. By selecting the “Infusion Pump and BGM Settings” 616, the user can turn on or off communications with an infusion pump and/or a BGM. In addition, in some embodiments the user can select the type of infusion pump and/or BGM being used in the system. In particular embodiments, the brand and model number of the infusion pump and/or BGM can be selected. When the system is so configured with the type, brand, or brand and model number of infusion pump and/or BGM being used in the system, the communications with the infusion pump and/or BGM, and the controls thereof, can be enhanced.

The user may also select “Wireless Settings” 618 from the “communication Settings” category. For example, the type of wireless communication interface to be used for communications with other components of the user's physiological sensing system can be configured. Such other components can include, but are not limited to, an infusion pump, a BGM, the user's computing device, an alarm clock, a base station, an emergency call center, and the like. For example, Bluetooth, RF, WiFi, cellular phone communications, and other types of wireless communications can be selected as the type of wireless communication interface to be used for communications with the various components. The user may configure different types of wireless communication for the different components of the user's physiological sensing system as desired.

Referring again to FIG. 6A, when a selection of “Alarm Settings” 630 is made, in some embodiments the configuration of FIG. 6C is presented on the touchscreen display 122 of the user's computing device 120. The selections presented in the category of “Alarm Settings” can include, but are not limited to, “Alarm Tones” 632, “Vibration Alarm” 634, “Non-wearing Alarms” 636, and “Caregiver and 911 Alarms” 638.

Referring now to FIG. 6C, the user can select “Alarm Tones” 632 to configure the alarm tones to be used for the user's physiological sensing system. “Alarm tones” refer to the sounds to be emitted by the user's wearable physiological sensing device 110 and/or computing device 120 when an alarm condition is deemed to exist. For example, the sounds may be rings, chimes, bells, buzzers, and so on. The user may configure particular types of alarm sounds to be used for particular types of alarm conditions. In some embodiments, the alarm tones may be voice synthesized annunciations of the type of alarm, such as “hypoglycemia event detected.” In some embodiments, different levels of alarms may be configured to initiate different types of alarm sounds. For example, the user may configure a moderate alarm level to initiate a bell sound, whereas the user may configure a severe alarm level to initiate a loud buzzer sound.

The user can select “Vibration Alarm” 634 to configure the tactile alarms to be used for the user's physiological sensing system. For example, the user may choose to activate a vibration alarm to be initiated at the wearable physiological sensing device 110 in the event of all or just particular types of alarm events. The vibration alarms can be configured to be provided in combination with alarm tones, or without alarm tones being emitted.

Still referring to FIG. 6C, the user can select “Non-wearing Alarms” 636 to configure the alarm settings for alarm events relating to non-use of the wearable physiological sensing device 110. In other words, the wearable physiological sensing device 110 can detect when the user is not wearing the sensing device 110, and one or more alarms can be initiated in accordance with the configured settings of the “Non-wearing Alarms” 636. In some embodiments, the skin temperature sensor of the wearable physiological sensing device 110 can be used to determine whether the user is wearing the sensing device 110. In other embodiments, other sensors, such as a perspiration sensor, can be used to determine whether the user is wearing the sensing device 110.

By selecting the “Non-wearing Alarms” 636 settings, the user may configure settings such as a threshold period of time of non-use which, if exceeded, will result in the initiation of an alarm. For example, the user may configure the settings of the “Non-wearing Alarms” 636 such that an alarm will be initiated if the user is not wearing the wearable physiological sensing device 110 for more than a threshold period of time, which in some embodiments may be about 30 minutes, one hour, two hours, more than two hours, or any time period therebetween. Different threshold time periods may also be established for different times of day. For example, the user may configure a first threshold period of time for the nighttime, and a different threshold period of time for the daytime. The user may also configure the type of alarms to be initiated, and to which devices of the user's physiological sensing system such alarms are to be sent. For example, the user may configure the settings of the “Non-wearing Alarms” 636 so that if the nighttime threshold time period for non-use is exceeded, a SMS text message is sent to the primary caregiver. In another example, the user may configure the settings of the “Non-wearing Alarms” 636 so that if a threshold time period for non-use is exceeded, first an alarm is initiated at the wearable physiological sensing device 110 and/or the user's computing device 120. Then, if the sensing device 110 is still not being worn after another period of time during which the sensing device 110 and/or the user's computing device 120 are alarming, an alarm can be sent to the primary caregiver. Similarly, an alarm can be sent to the secondary caregiver if the sensing device 110 is still not being worn within another period of time after which the alarm was sent to the primary caregiver.

The user can select “Caregiver and Emergency Response Alarms” 638 to configure the alarms to be sent to the caregivers and the emergency call center. The emergency call center can be a remote emergency monitoring and dispatch service, a governmental “9-1-1” call center, and the like. The user can configure the mode of communication to be used when alarms or messages are sent to these members of the user's physiological sensing system. The communications can be sent using modalities such as a cellular telephone network, land-based telephone network, internet, and other modes and combinations thereof. For example, the user may configure the settings so that alarms sent to the primary caregiver are sent by SMS text message, and to the secondary caregiver by email. Or, the alarms sent to the caregivers may trigger an alarming function of an application associated with the user's physiological sensing system being run on a computing device of the caregiver(s). The user may also configure the mode of communications to the emergency call center. For example, the user may choose to configure communications to the emergency call center as a telephone call to “911.” Or, the user may choose to configure communications to the emergency call center as a SMS text message (or email, or telephone call) to a non-governmental remote emergency monitoring and dispatch service.

Referring again to FIG. 6A, when a selection of “Sensor Settings” 650 is made, in some embodiments the configuration of FIG. 6D is presented on the touchscreen display 122 of the user's computing device 120. The selections presented in the category of “Sensor Settings” can include, but are not limited to, “Skin Temp and Perspiration On/Off and Sensitivity” 652, “Oximeter Settings” 654, “Pedometer Settings” 656, and “GPS Settings” 658.

Referring now to FIG. 6D, the user can select “Skin Temp and Perspiration On/Off and Sensitivity” 652 to configure settings to be used for the skin temperature and perspiration systems of the user's physiological sensing system. For example, the user may activate or deactivate the skin temperature and/or perspiration systems. In addition, the user may increase or decrease the sensitivity of the skin temperature and/or perspiration systems, and adjust the alarm threshold limit values. In some embodiments, such settings may be protected by security (such as a PIN or password), and only a caregiver or medical personnel may make such setting adjustments. By making such adjustments, the user's wearable physiological sensing device 110 can be customized to the user's physiological characteristics, to reduce false alarms and to enhance the overall effectiveness of the user's physiological sensing system.

The user can select “Oximeter Settings” 654 to configure settings to be used by the oximetry system of the user's physiological sensing system. For example, the user can activate or deactivate the functionality of the oximetry system, such as detection of the user's pulse rate and oxygen saturation. In some embodiments, alarm threshold limit values for the parameters of the oximetry system can also be set using the “Oximeter Settings” 654.

The user can select “Pedometer Settings” 656 to configure settings to be used by the pedometer system of the user's physiological sensing system. For example, the user can activate or deactivate the functionality of the pedometer system, such as detection of the user's activity level. In some embodiments, the user can also set whether the activity level as determined by the pedometer system is to be communicated to an infusion pump system to be used as an input for a determination of insulin dispensation amounts.

Still referring to FIG. 6D, the user can select “GPS Settings” 658 to configure settings to be used by the GPS system of the user's physiological sensing system. For example, the user can activate or deactivate the functionality of the GPS system. In addition, for GPS systems that are active GPS tracking systems, an address to periodically communicate the locational coordinates information to can be set.

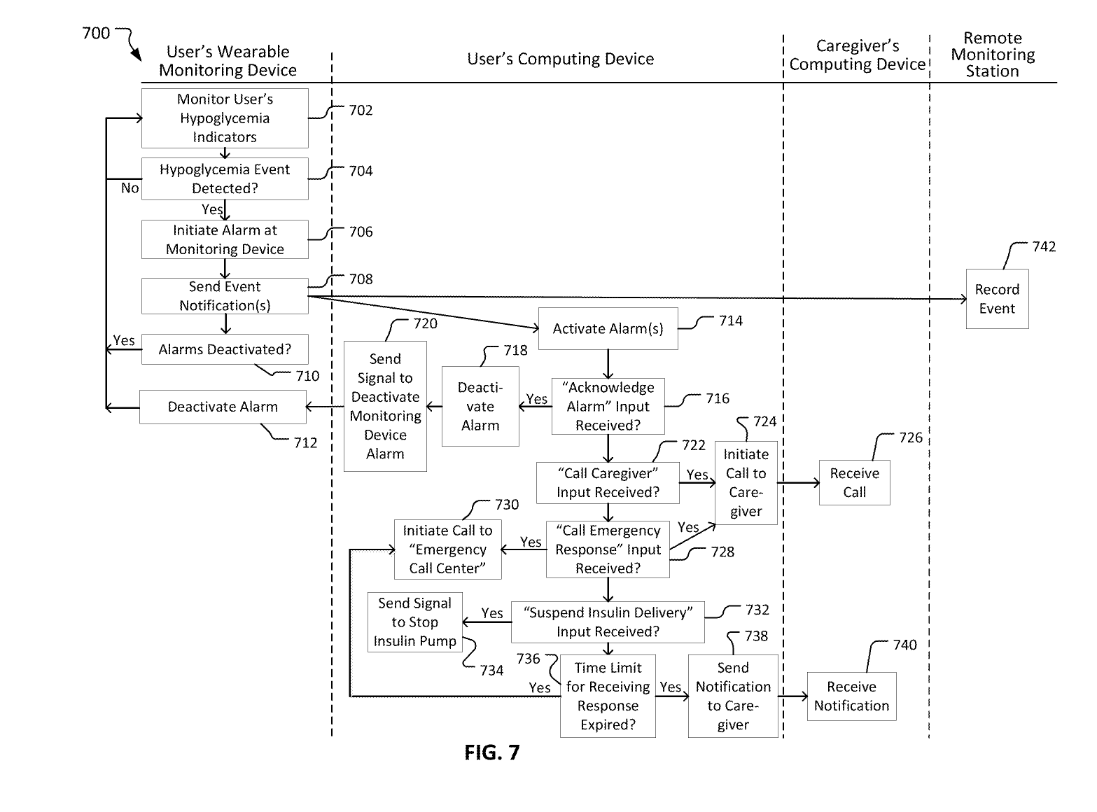

Referring now to FIG. 7, a diagram is provided to illustrate an example process 700 by which the system 100 of FIG. 1 can operate in accordance with some embodiments. In this example, the operations of (1) the user's wearable sensing device 110, (2) the user's computing device 120, (3) the caregiver's computing device 180, and (4) the remote monitoring station 190 are described. It should be understood that the operations of a physiological sensing system is dependent on, for example, the types and configurations of the devices included in the physiological sensing system, as well as the user settings that have been selected (e.g., refer to FIGS. 6A-6D). In this example, measurement of hypoglycemia indicators and communication of associated alarms are used to illustrate the operations of the physiological sensing system. However, it should be understood that the process 700 is also illustrative regarding the monitoring and alarming of other types of physiological parameters that are measurable using the physiological sensing systems provided herein.

At operation 702, the user's hypoglycemia indicators are monitored using a system. For example, as described above in reference to FIG. 2, multiple sensors of a system can detect physiological indicators of hypoglycemia. Such sensors can include a perspiration sensor, a skin temperature sensor, and a graphene-based glucose sensor. Such monitoring can occur periodically, such as about every second, about every other second, about every third or fourth second, and so on.

At operation 704, the system determines whether a hypoglycemia event has been detected. This operation can be performed using the monitored hypoglycemia indicators from operation 702. The monitored hypoglycemia indicators can be used as inputs to algorithms that calculate values and compare the calculated values to threshold limit values. The algorithmic calculations can be performed in the wearable monitoring device, or in a combination of the wearable monitoring device and the user's computing device. If the comparison of the calculated values to the threshold limit values results in a determination that no hypoglycemia event has been detected, the process 700 returns to operation 702. However, if the comparison of the calculated values to the threshold limit values results in a determination that a hypoglycemia event has been detected, the process proceeds to operation 706.

At operation 706, an alarm is initiated at the wearable physiological sensing device. The alarm can be an audible alarm, a visual alarm, a tactile alarm, or a combination thereof. In some embodiments, the severity of the hypoglycemia event is signified by the type of alarm. For example, a visual alarm may be colored-coded in particular ways to signify the severity of the hypoglycemia event. Audible alarms, may use a particular type of tone, or the volume of the alarm may be adjusted to signify the severity of the hypoglycemia event. The alarms are configurable according to user preferences as described above.

At operation 708, an event notification is sent to other devices within the physiological sensing system. In this example embodiment, an event notification is sent to both the user's computing device and to a remote monitoring station. The techniques for sending such event notifications are described above in reference to FIG. 1, for example. While in this example, the notification to the remote monitoring station is sent by the wearable monitoring device, in some embodiments the notification to the remote monitoring station is sent by the user's computing device. At operation 742, the remote monitoring station stores the event notification in a database for future analysis. The remote monitoring stations may be, for example, a health clinic, doctor's office, hospital, a research facility, or another organization used for tracking user physiological parameters. The stored record can include a time stamp indicating when the event occurred. As described above in reference to FIG. 5, in some embodiments the user may append the record of the event notification stored at the remote monitoring station with contextual information by selecting an option to do so.

At operation 714, an alarm is activated at the user's computing device upon receipt of the event notification sent by the user's wearable monitoring device in operation 708. The alarm at the user's computing device can be an audible alarm, a visual alarm, a tactile alarm, or a combination thereof. In some embodiments, the severity of the hypoglycemia event is signified by the type of alarm. For example, a visual alarm may be colored-coded in particular ways to signify the severity of the hypoglycemia event. Audible alarms, may use a particular type of tone, or the volume of the alarm may be adjusted to signify the severity of the hypoglycemia event. The alarms are configurable according to user preferences as described above.

At operation 710, the user can deactivate the alarms at the wearable monitoring device and user's computing device using the user interface of the wearable monitoring device. This technique for responding to the alarms may be convenient for the user because of the accessibility of the wearable monitoring device to the user. In some embodiments, the deactivation of the alarms (also referred to herein as an acknowledgement of the alarm) at the wearable monitoring device can be performed using a button, a touchscreen display, a voice command, a gesture, and the like, or combinations thereof. After deactivating the alarms in this operation 710, the process 700 returns to operation 702 where further monitoring of the user's hypoglycemia indicators is performed. If no alarm deactivation is enacted at operation 710, the alarms at the wearable monitoring device and user's computing device continue to be manifested.

At operation 716, the user has the option to acknowledge the alarm using the user's computing device. This can be performed, for example, by the selection of a user input that is presented on the display of the user's computing device (refer, e.g., to FIG. 5). If no such acknowledgement of the alarm is received at the user's computing device, the alarms will continue to be manifested at the user's computing device and the wearable monitoring device. However, when an acknowledgement of the alarm is received at operation 716, the process 700 proceeds to operation 718. At operation 718, the alarm that is manifesting at the user's computing device is deactivated in response to the user acknowledgement received at operation 716. In addition, at operation 720, the user's computing device sends a signal to the user's wearable monitoring device to deactivate the alarm at the wearable monitoring device. At operation 712, in response to receiving the signal from the user's computing device to deactivate the alarm at the wearable monitoring device, the alarm at the wearable monitoring device is deactivated. Then the process 700 continues to perform further monitoring of the user's hypoglycemia indicators at operation 702.

At operation 722, the user has the option to initiate a telephone call to the user's designated caregiver using the user's computing device. This can be performed, for example, by the selection of a user input that is presented on the display of the user's computing device (refer, e.g., to FIG. 5). If such a selection is made by the user, a telephone call is initiated to the caregiver from the user's computing device in operation 724. In operation 726, the caregiver's computing device receives the telephone call from the user's computing device. Then the user and the caregiver can converse regarding the hypoglycemia event alarm. For example, the user may request assistance from the caregiver. In some embodiments, a primary caregiver and a secondary caregiver may be designated by the user (e.g., refer to FIG. 6B). In some such embodiments, if the primary caregiver has not received the telephone call initiated at operation 724 after a particular number of rings (which can be a configurable number of rings), then a telephone call to the secondary caregiver may be automatically initiated by the user's computing device.

At operation 728, the user has the option to initiate a telephone call to the user's designated emergency call center using the user's computing device. This can be performed, for example, by the selection of a user input that is presented on the display of the user's computing device (refer, e.g., to FIG. 5). If the user selects this option, a call is initiated by the user's computing device to the designated emergency call center at operation 730. The user's preferred emergency call center can be designated in the settings of the user's computing device such as described in reference to FIG. 6C.

At operation 732, the user has the option to suspend insulin deliveries using the user's computing device. This can be performed, for example, by the selection of a user input that is presented on the display of the user's computing device (refer, e.g., to FIG. 5). As described above in reference to FIG. 1, in some embodiments the user's system includes an infusion pump that is in communication with the wearable monitoring device and/or the user's computing device. If the user selects this option, a signal to stop dispensations of insulin from the infusion pump is sent from the user's computing device to the infusion pump at operation 734. In alternative embodiments, the signal to the infusion pump can originate from the wearable monitoring device. In either case, upon receipt of the signal, the infusion pump suspends insulin dispensations. Thereafter, a periodic notification can be provided to the user via the wearable monitoring device and/or the user's computing device to remind the user that insulin dispensations from the infusion pump have been suspended. Such reminders can prompt the user to reactivate the infusion pump at the appropriate time.

At operation 736, the user's computing device monitors whether a user input has been received within a threshold period of time after the initiation of the hypoglycemia alarm event. The threshold period of time can be a user configurable amount of time, such as one minute, two minutes, three minutes, four minutes, five minutes, and so on. If no user input has been received within the threshold period of time after the initiation of the hypoglycemia alarm event, a notification is sent to the caregiver at operation 738. At operation 740, the notification is received by the caregiver's computing device. This notification can be an alarm, a telephone call, a SMS text message, and the like. The notification to the caregiver can prompt investigative actions by the caregiver to determine whether the user needs assistance. If a user input was received by the wearable monitoring device or the user's computing device within the threshold period of time after the initiation of the hypoglycemia alarm event, no such notification is sent to the caregiver.

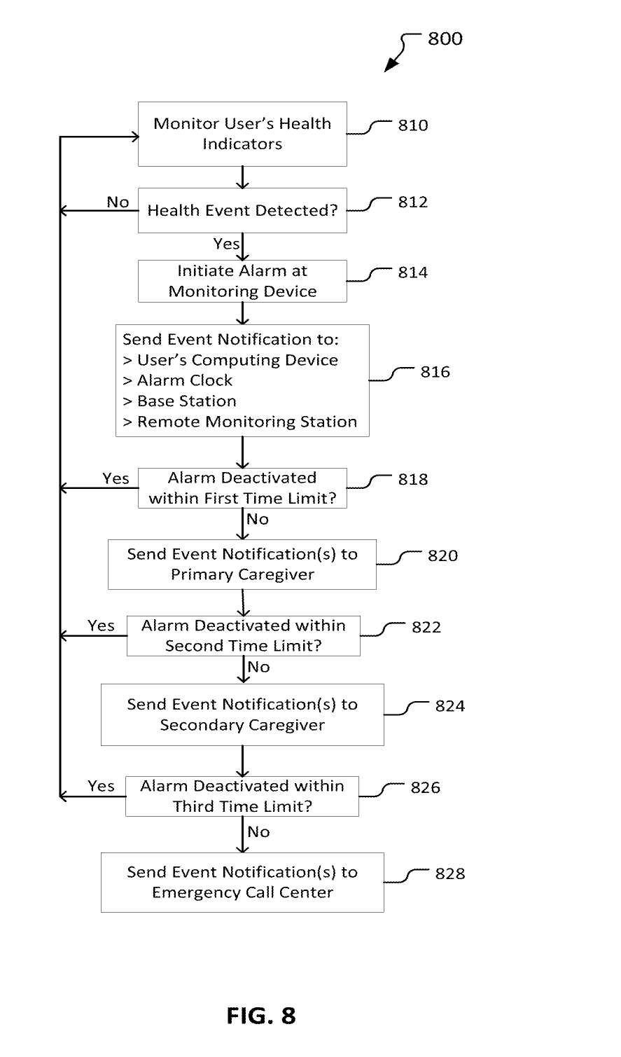

Referring now to FIG. 8, a wearable physiological sensing device can perform a process 800 for monitoring a user's physiological parameters, and for notifying other devices within a physiological sensing system of alarm conditions. In some embodiments of process 800, the wearable physiological sensing device can be configured as described above in reference to FIG. 2. The alarm event notifications can be sent to other devices, such as the devices described above in reference to FIG. 1. However, it should be understood that the other devices are optional and some or all of such other devices may not be included in the user's physiological sensing system.

At operation 810, the wearable physiological sensing device monitors the user's health indicators. As described above in reference to FIG. 2, such health indicators can include, but are not limited to, perspiration, skin temperature, pulse rate, oxygen saturation of the user's blood, blood glucose level, movement, and activity level.

At operation 812, the wearable physiological sensing device determines whether a health event is detected (e.g., a hypoglycemia event or another health event). In some embodiments, the sensing device can make such a determination by using the monitored health indicator values as inputs to one or more algorithms, and comparing the results of the algorithms to threshold limit values. If no health event is detected, the process 800 returns to operation 810 and monitoring of the user's health indicators continues. However, if a health event is detected, the process proceeds to operation 814 where an alarm is initiated at the wearable physiological sensing device. The alarm can be an audible alarm, a visual alarm, a tactile alarm, or a combination thereof. In some embodiments, the severity of the health event is signified by the type of alarm. For example, a visual alarm may be colored-coded in particular ways to signify the severity of the health event. Audible alarms, may use a particular type of tone, or the volume of the alarm may be adjusted to signify the severity of the health event. The alarms are configurable according to user preferences as described above in reference to FIG. 6C.

At operation 816, the wearable physiological sensing device initiates communications regarding the health event to be sent to other devices and sub-systems within the user's physiological sensing system. For example, some of the devices and sub-systems within the user's physiological sensing system can include, but are not limited to, a computing device of the user, an alarm clock, a base station, and a remote monitoring station. In some embodiments, the wearable physiological sensing device sends a notification to one of the devices and then that device relays the notification to another device. For example, a base station may receive a notification from the wearable physiological sensing device, and then the base station may relay that notification to a remote monitoring station. Other such combinations for relaying notifications are also envisioned within the scope of this invention. The communications may be in various forms (alarms, emails, telephone calls, SMS text messages, automated voice messages, and the like), and may be transmitted via various modes of communication and combinations of such modes of communication (cellular telephony, landline telephony, WiFi, internet, intranet, Bluetooth, RF, and the like).

At operation 818, the wearable physiological sensing device monitors the time that expires after the initiation of the alarm and before receiving a user input to deactivate the alarm. If a user input to deactivate the alarm is received after the initiation of the alarm and before the end of a first threshold period of time, the process 800 returns to operation 810 where monitoring of the user's health indicators continues. However, when no user input to deactivate the alarm is received before the expiration of the first threshold period of time, at operation 820 the wearable physiological sensing device initiates a notification of the alarm event to be sent to the user's designated primary caregiver. As with the communications described above, the notification to the primary caregiver may be in various forms, and may be transmitted via various modes of communication and combinations of such modes of communication.

At operation 822, the wearable physiological sensing device monitors the time that expires after the initiation of the notification to the user's primary caregiver and before receiving a user input to deactivate the alarm. If a user input to deactivate the alarm is received after the initiation of the alarm and before the end of a second threshold period of time, the process 800 returns to operation 810 where monitoring of the user's health indicators continues. However, when no user input to deactivate the alarm is received before the expiration of the second threshold period of time, at operation 824 the wearable physiological sensing device initiates a notification of the alarm event to be sent to the user's designated secondary caregiver. As with the communications described above, the notification to the secondary caregiver may be in various forms, and may be transmitted via various modes of communication and combinations of such modes of communication.

At operation 826, the wearable physiological sensing device monitors the time that expires after the initiation of the notification to the user's secondary caregiver and before receiving a user input to deactivate the alarm. If a user input to deactivate the alarm is received after the initiation of the alarm and before the end of a third threshold period of time, the process 800 returns to operation 810 where monitoring of the user's health indicators continues. However, when no user input to deactivate the alarm is received before the expiration of the third threshold period of time, at operation 828 the wearable physiological sensing device initiates a notification of the alarm event to be sent to the user's designated emergency call center. As with the communications described above, the notification to the emergency call center may be in various forms, and may be transmitted via various modes of communication and combinations of such modes of communication.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Accordingly, other embodiments are within the scope of the following claims.

What is claimed is:

1. A wearable physiological sensing device comprising:
a housing that is configured to be worn by a user such that a portion of the housing is in contact with a skin surface of the user;
a perspiration sensor system disposed at least partially within the housing and configured to detect a level of perspiration on the skin surface of the user;
a temperature sensor system disposed at least partially within the housing and configured to detect a temperature of the skin surface of the user;
control circuitry disposed at least partially within the housing and configured to detect a hypoglycemic event of the user in response to receiving sensor information from the perspiration sensor system indicative of the level of perspiration on the skin surface of the user and from the temperature sensor system indicative of the temperature of the skin surface of the user;
a user interface attached to the housing and configured to an output alarm when the control circuitry detects the hypoglycemic event of the user; and
a wireless communication device disposed at least partially within the housing and configured to wirelessly communicate with one or more external devices.

2. The wearable physiological sensing device of claim 1, comprising a GPS system at least partially within the housing.

3. The wearable physiological sensing device of claim 1, comprising a pedometer system at least partially within the housing.

4. The wearable physiological sensing device of claim 1, comprising one or more accelerometers at least partially within the housing.

5. The wearable physiological sensing device of claim 1, comprising an oximetry system at least partially within the housing.

6. The wearable physiological sensing device of claim 1, comprising an inductive battery charging system at least partially within the housing.

7. The wearable physiological sensing device of claim 1, comprising a graphene-based glucose sensor system at least partially within the housing.

8. The wearable physiological sensing device of claim 1, wherein the wireless communication device utilizes Bluetooth.

9. The wearable physiological sensing device of claim 1, wherein the wearable physiological sensing device is configured to wear on a wrist of the user.

10. The wearable physiological sensing device of claim 1, wherein the housing is configured to engage with a docking station.

11. A wearable physiological sensing and response system, the system comprising:
a wearable physiological sensing device comprising:
a housing that is configured to be worn by a user such that a portion of the housing is in contact with a skin surface of the user;
a physiological sensor positioned at least partially within the housing and configured to detect a physiological parameter of the user via contact with the skin surface of the user; and
a wireless communication device disposed at least partially within the housing and configured to wirelessly communicate with one or more external devices; and
a computing device that is separate from the wearable physiological sensing device and that is configured to wirelessly communicate with the wireless communication device such that the computing device is configured to provide notifications of physiological alarm events in response to receiving an alarm event communication from the wireless communication device of the wearable physiological sensing device.

12. The system of claim 11, wherein the physiological sensor comprises a perspiration sensor system or a temperature sensor system.

13. The system of claim 11, wherein the computing device is a smartphone.

14. The system of claim 13, wherein the wireless communication device and the smartphone are configured to communicate using Bluetooth.

15. The system of claim 11, wherein the computing device is configured to wirelessly provide notifications of physiological alarm events in response to receiving an alarm event communication from the wireless communication device of the wearable physiological sensing device to a second computing device.

16. The system of claim 15, wherein the computing device is configured to wirelessly provide the notifications of physiological alarm events in response to receiving an alarm event communication from the wireless communication device of the wearable physiological sensing device to a second computing device using WiFi.

17. The system of claim 15, wherein the computing device is configured to wirelessly provide the notifications of physiological alarm events in response to receiving an alarm event communication from the wireless communication device of the wearable physiological sensing device to a second computing device via a SMS text message.

18. A method for monitoring a user's physiological parameters, the method comprising:
placing a wearable physiological sensing device in contact with a skin surface of the user, wherein the wearable physiological sensing device comprises:
a housing that is configured to be worn by the user such that a portion of the housing is in contact with the skin surface of the user;
a perspiration sensor system disposed at least partially within the housing and configured to detect a level of perspiration on the skin surface of the user;
a temperature sensor system disposed at least partially within the housing and configured to detect a temperature of the skin surface of the user; and
a wireless communication module disposed at least partially within the housing and configured to send and receive wireless communications to and from one or more other devices;
monitoring the level of perspiration on the skin surface of the user and the temperature of the skin surface of the user;
determining, based on the monitored level of perspiration on the skin surface of the user and the monitored temperature of the skin surface of the user, whether a physiological alarm condition exists;
initiating, based on a determination that a physiological alarm condition exists, a first alarm notification at the wearable physiological sensing device, and sending a wireless alarm event communication from the wireless communication module;
receiving, at a second device that is not connected to the wearable physiological sensing device, the alarm event communication sent from the wireless communication module; and
providing, in response to receiving the alarm event communication sent from the wireless communication module, a second alarm notification at the second device.

19. A wearable physiological sensing device comprising:
a wearable housing including an exterior surface configured to contact with a skin surface of the user;
at least one physiological parameter sensor positioned at least partially within the housing and configured to detect physiological parameter via the skin surface of the user;
control circuitry disposed at least partially within the housing and configured to detect a health alarm event of the user in response to receiving sensor information from the physiological parameter sensor;
a user interface attached to the housing and configured to an output alarm when the control circuitry detects the health alarm event; and
a wireless communication device mounted at least partially within the housing and configured to wirelessly communicate with one or more external devices.

20. A system comprising:
wearable physiological sensing device, including:
a wearable housing including an exterior surface configured to contact with a skin surface of the user;
at least one physiological parameter sensor positioned at least partially within the housing and configured to detect physiological parameter via the skin surface of the user;
a wireless communication device mounted at least partially within the housing and configured to wirelessly communicate with one or more external devices
wherein wearable physiological sensing device is configured to detect a health alarm event of the user in response to receiving sensor information from the physiological parameter sensor; and
a computing device that is separate from the wearable physiological sensing device and that is configured to wirelessly communicate with the wireless communication device, wherein the computing device outputs notifications of physiological alarm events in response to receiving an alarm event communication from the wireless communication device of the wearable physiological sensing device.
